(19) United States
(12) Patent Application Publication
(10) Pub. No.: US 2026/0365956 A1
(43) Pub. Date: Nov. 19, 2026

(54) MODULAR MACROFLUIDIC CELL DEVICE

(71) Applicant: KENT STATE UNIVERSITY, Kent, OH (US)

(72) Inventors: Elda HEGMANN, Kent, OH (US); Abraham VALERIO-AGUILAR, Kent, OH (US); Gisele PEGORIN, Kent, OH (US); Faeze SHIRALIPOUR, Kent, OH (US); Yeganeh NIKAKHTAR, Kent, OH (US); Ashley GILMOR, Wooster, OH (US); Robert CLEMENTS, Kent, OH (US)

(21) Appl. No.: 19/681,223

(22) Filed: May 19, 2026

Related U.S. Application Data
(60) Provisional application No. 63/808,092, filed on May 19, 2025.

(51) Int. Cl. C12M 3/00; C12M 1/00; C12M 1/12

(52) U.S. Cl. CPC C12M 23/44; C12M 23/22; C12M 23/46; C12M 25/14

(57) ABSTRACT

A modular macrofluidic cell device including an internal, three-dimensional chamber especially suited for three-dimensional cell culture, research, experimentation and observation. The cell device is highly adaptable and has the ability to house modular implants having a myriad of configurations within the chamber allowing experiments to be conducted in environments that mimic those found in the body. Still further, the cell device is readily transportable, for example between various laboratory stations, such as an incubator chamber and testing station. In addition the cell device is both reusable and recyclable.

### FIELD OF THE INVENTION

The present invention relates a modular macrofluidic cell device including an internal, three-dimensional chamber especially suited for three-dimensional cell culture, research, experimentation and observation. The cell device is highly adaptable and has the ability to house modular implants having a myriad of configurations within the chamber allowing experiments to be conducted in environments that mimic those found in the body. Still further, the cell device is readily transportable, for example between various laboratory stations, such as an incubator chamber and testing station. In addition the cell device is both reusable and recyclable.

### BACKGROUND OF THE INVENTION

The biotechnology, chemical, pharmaceutical and related industries have developed various cell culture platforms as systems having environments for studying and/or growing cells in vitro. The known cell culture platforms have applications in cell study, drug development, drug screening and tissue engineering for example.

Traditional two-dimensional cell culture platforms, for example petri dishes, while being easy to use and relatively inexpensive, provide unnatural and unrealistic cell environments that often produce biologically irrelevant experimental results.

The use of animal testing for cell culture is relatively complex and also ethically controversial.

Some 3D cell culture platforms including macrofluidic devices are available, but can be relatively complex and expensive to utilize.

In view of the above, the art still needs devices that bridge the gap in cell culture technology by using a low-cost, macrofluidic cell device for three-dimensional cell culture.

### SUMMARY OF THE INVENTION

The problems noted above and others are solved by the present invention which provides a customizable, modular macrofluidic cell device.

In an embodiment of the present invention, the macrofluidic cell device is highly customizable and cost efficient which enables study of cell cultures in a three-dimensional, controlled environment designed to replicate one or more realistic biological conditions.

In a first aspect or embodiment, the modular macrofluidic cell devices of the present invention include a main body having an internal, three-dimensional chamber having a volume capable of housing a sample to be studied.

Importantly, in one aspect or embodiment the cell devices of the present invention have customizable inlet ports and outlet ports in various walls, sides or surfaces of a main body containing a three-dimensional chamber so different kinds of attachments can be removably connected to the cell devices and fluids such as, but not limited to, a growing media, air, or drugs can be introduced into the chamber at desired rates. Also, the number of inlet ports and outlet ports can be modified to the specific requirements of each experiment or use case.

In an additional aspect or embodiment, the cell device includes a pillar that can optionally be utilized as a support for a sample to be studied in the cell device. The pillar is additionally and/or alternatively utilized to focus a light beam to the inside of the chamber, preferably on the sample, due to the presence of a light path located within the pillar. Light beams are provided by a suitable external light source.

In yet an additional aspect or embodiment of the invention, the cell device includes at least one window surface that is optically transparent which allows observation and/or study of the chamber and sample located therein with an optical device such as a microscope. In a highly preferred embodiment, the device includes a first window surface opposite a second window surface, both being optically transparent whereby light is able to pass through the cell device via the window surfaces.

In still a further aspect or embodiment of the invention, the cell device includes a hatch operatively and removably connected to the main body to allow placement of the sample into the chamber and removal of the sample from the chamber as necessary or desired. In a preferred embodiment, the hatch includes one of the window surfaces that is optically transparent.

Still another aspect or embodiment of the present invention includes a modular implant which is insertable into and removable from the chamber, for example through one or more of the hatch, the at least one inlet port, and the at least one outlet port. The modular implant can be designed in many different forms for example a scaffold, in particular having the form of a vascular network, a neural network, an optical network, or the like. Having such a modular design permits 3D printed constructs or organoids to be easily attached and/or plugged into the chamber. In addition, if the organoids/models have an imbedded vascular system, the vascular system’s inputs and outputs can be attached to the modular implant inlet and outlet to increase perfusion within grown samples. The cell device platforms of the present invention permit long term growth of organotypic and other culture systems. For example, a 3D printed modular implant, such as for example, a kidney or fabricated scaffold to grow bone cells, can be attached within the chamber of the device for studying tissues in vitro. Macrofluidic implants or inserts are also able to contain their own vascular system, whereby artificial vessels permeate throughout the insert and inputs/outputs are attached to the media flow using the modular attachment system. This helps alleviate one challenge of larger culture systems; adequate waste removal and nutrient delivery to the center of the construct permitting longer experiments and more accurate results.

In an additional aspect or embodiment of the present invention, the cell device inlet port(s) and outlet port(s) through which fluid can flow can be structurally arranged on the main body to provide customized flow paths, for example linear flow, branch flow, and also provides the ability to allow fluids to be combined with each other, the ability to divert fluids, the ability to cause laminar flow between fluids, and to dilute fluids. Customized designed conduits can provide a method to direct fluid flow through using these paths to enhance sample perfusion.

In still a further aspect or embodiment, the macrofluidic cell device includes at least one inlet port having at least one inlet channel adapted to carry at least one continuous phase fluid and at least one outlet port having a channel adapted to discharge at least one continuous phase fluid from the device. The three-dimensional chamber is in fluid communication between the at least one inlet port and the at least one outlet port. A fluidic nozzle is adapted to be connected to the inlet port for flowing a fluid through the cell device. The use of multiple inlet ports can be utilized to disperse multiple different fluids which are either miscible or immiscible with each other.

In still a further aspect or embodiment, the main body of the macrofluidic cell device can be 3D printed, either fully or partially, so that the three-dimensional chamber therein has a desired volume and structure. For example, three-dimensional chambers have shapes in the form of a cube, a cuboid, a cylinder, a sphere or any other desired geometrical shape.

In yet a further aspect or embodiment, the one or more inlet ports and one or more outlet ports are formed as male fittings including an aperture which can be capped in order to isolate the three-dimensional chamber, and any fluid therein, from the environment. The device is thereby readily portable and can be transported between any two locations. For example, the macrofluidic cell device can be easily transferred between an incubator and an imaging station. The structure of the device also allows imaging to be performed while fluid is flowing through the chamber or while no fluid is running through the chamber.

For the avoidance of doubt, it is understood that while various embodiments or aspects of the invention are described individually, it should be clear that two or more embodiments or aspects can be, and often times are, present in a single device according to the present invention.

### BRIEF DESCRIPTION OF THE DRAWINGS

The invention will be better understood and other features and advantages will become apparent by reading the detailed description of the invention, taken together with the drawings, wherein:

FIG. 1 is a top perspective view of one embodiment of a macrofluidic cell device of the present invention;

FIG. 2 is a top view thereof;

FIG. 3 is a top perspective view of one embodiment of a macrofluidic cell device of the invention including an inlet port and an outlet port located in opposite walls of the main body;

FIG. 4 is a top perspective view of one embodiment of a macrofluidic cell device of the invention including two inlet ports in the same wall and an outlet port located in an opposite wall of the main body;

FIG. 5 is a top perspective view of one embodiment of a macrofluidic cell device of the invention including an inlet port and an outlet port located in the same wall of the main body;

FIG. 6 is a top perspective view of a second embodiment of a microfluidic cell device of the invention illustrating the hatch having a frame shown in darker shading as compared to the main body;

FIG. 7 is a perspective view of FIG. 6 wherein the hatch has been partially opened;

FIG. 8 illustrates the second embodiment of the microfluidic cell device with the hatch separated from the main body;

FIG. 9 is a partial cross-section view of a further embodiment of the microfluidic cell device of the invention illustrating a light path through a pillar of the device;

FIG. 10A illustrates a side view of one embodiment of a modular implant in the shape of a vascular system;

FIG. 10B illustrates a front view of the outlets of the vascular system modular implant illustrated in FIG. 10A;

FIG. 11 illustrates a top view of a microfluidic cell device having a modular implant operatively connected to an inlet port of the main body, with the modular implant being formed as a vascular system;

FIGS. 12A-12D illustrate the flow of a fluid through a microfluidic cell device of the present invention having a sample located therein that is connected to a pillar, wherein FIG. 12A illustrates fluid beginning to enter the interior chamber of the main body from the right side, FIG. 12B illustrates the liquid filling the chamber cavity, FIG. 12C illustrates the fluid completely filling the cavity and FIG. 12D illustrates the fluid exiting the microfluidic device through the outlet port on the left side thereof.

### DETAILED DESCRIPTION

Detailed embodiments of the macrofluidic cell devices of the present invention and methods for using the same are disclosed herein. However, it is to be understood that the disclosed embodiments are merely exemplary of the present devices and methods may be embodied in various forms. Therefore, specific functional details disclosed herein are not to be interpreted as limiting, but merely as a basis of the claims and as a representative basis for teaching one of ordinary skill in the art to variously employ and construct the apparatuses and utilize the methods for producing and using the cell devices.

For purposes of this disclosure, the term “in fluid communication” or “fluidly connected” is intended to define components or parts of the same components that are operatively interconnected to allow fluid flow between components or parts of components.

For purposes of this disclosure, the term “sample” and similar terms should be understood to mean any construct suitable for use within the cell device, and preferably a biologically-derived construct, for example a cellular component.

Purposes of this disclosure, the term “optically transparent” is intended to define a structure, particularly a surface of the macrofluidic device, that allows an observation to be made by an optical device located on the outside of the cell device of a sample or other feature in the device chamber through the optionally transparent surface. For example, when considering a hatch or window surface of the macrofluidic cell device, an optical device positioned outside of the cell device can observe a sample located in the three-dimensional chamber due to the optical transparency of the hatch or window surface.

The present invention advantageously provides a self-contained, modular, macrofluidic cell device especially adapted for three-dimensional cell culture, research, experimentation and observation. The cell device is highly adaptable and is able to be utilized in conjunction with many different configurations of modular implants allowing experiments to be conducted in environments that mimic those found in the body. The cell device is easily transportable between laboratory stations facilitating ease of use.

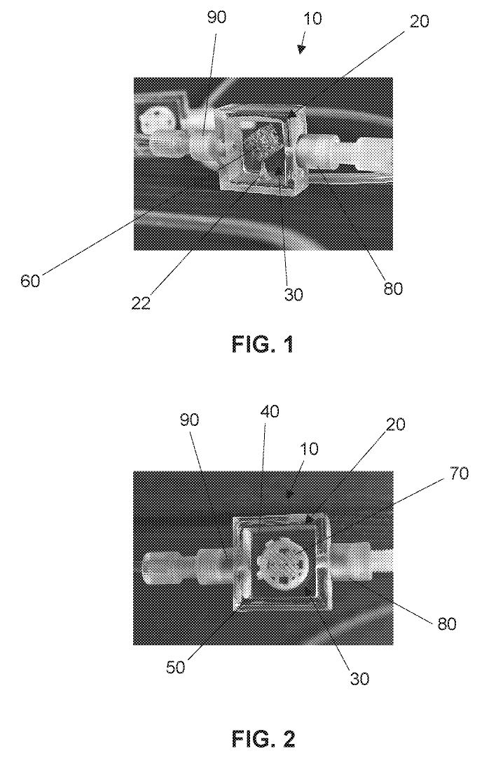

Referring now to the drawings wherein like reference numbers represent like parts throughout the several views, FIGS. 1 and 2 illustrate a macrofluidic cell device 10 having a main body 20 having an internal, three-dimensional chamber 30 having a volume. While main body 20 is essentially illustrated as a cube or cuboid, it should be readily understood that other configurations can be utilized. Similarly, while the chamber 30 illustrated is substantially in the shape of a cube, the main body can be fabricated so that the chamber assumes another form. Example chamber configurations include, but are not limited to, cubes, cuboids, cylinders, spheres, and prisms.

In a preferred embodiment, the main body is produced utilizing a 3D printer, which facilitates creating three-dimensional chambers of a desired volume and shape. Any polymer that can be 3D printed can be utilized to form the macrofluidic cell device, in particular the main body thereof. Preferred polymers are biodegradable and biocompatible with the fluids utilized in conjunction with the cell device and samples utilized therein. An example of a suitable 3D printer is commercially available from AnyCubic as Photon M3 Premium. Preferred polymers that can be utilized for forming the main body include, but are not limited to, commercially available printable polymers and resins or custom designed polymers and resins for specific tasks or experimental procedures. By way of nonlimiting examples, suitable polymers and resins include those that are photocurable or heat treatable.

The main body further includes a first window surface 40, which is the lower surface of the main body illustrated in FIG. 2. The first window surface is sufficiently optically transparent such that when the sample is present in the chamber at least one observation can be made of the sample 60 with an optical device through at least the first window surface. The cell device further includes a hatch 50, which is operatively and removably connected to the main body to allow placement of the sample 60 into the chamber and removal of the sample 60 from the chamber.

Preferably, as illustrated in FIGS. 1 and 2, hatch 50 is also optically transparent such that when the sample is present in the chamber at least one observation can be made of the sample with an optical device through the hatch and light can pass through the at least first window surface 40 and at least a portion of hatch 50.

It should be readily understood that the hatch may be located in a different position than that illustrated in FIG. 2, such as in a side wall. In such cases, the main body can include a second window surface, wherein the second window surface is sufficiently, optically transparent such that when the sample is present in the chamber at least one observation can be made of the sample with an optical device through both of the first and second windows, with the second window surface being opposite the first window surface.

Providing the cell device with at least two optically transparent sections positioned opposite of each other on different sides of the cell device allows use of the cell device with existing, commonly utilized optical systems, for example microscopes.

In preferred embodiments, a pillar 22 has a base connected to the main body 20 preferably integrally through 3D printing. The pillar 22 extends into the chamber and has a distal end to which the sample can be operatively connected in some embodiments. As illustrated in FIG. 9 pillar 122 has an internal light path 124 that extends from the base of the pillar to the distal, second end of the pillar located a distance from the base and positioned in the chamber. The light path is able to transmit light from a light source located outside of the chamber into the sample through the pillar 122 when located in the chamber for example as illustrated in FIG. 9.

The main body has at least one inlet port 80 extending through at least one wall or surface of the main body between an outer surface of the wall and the chamber for introducing a fluid into the chamber. The main body also includes at least one outlet port 90 extending through at least one wall of the main body between an outer surface of the wall and a chamber for allowing the fluid to exit the chamber. In a preferred embodiment, one or more of the at least one input port 80 and at least one outlet port 90 include a projection or fitting extending outwardly from the main body that can be capped to prevent fluid in the device from exiting the device through the respective inlet port and outlet port.

The inlet port 80 and outlet port 90 can be connected to any desirable fluid flow system.

In a further embodiment, the cell device includes a modular implant 70 for example as illustrated in FIG. 2. Modular implant 70 is insertable into and removable from the chamber, such as through hatch 50 or even inlet port 80 or outlet port 90. The modular implant 70 can serve as a substrate for the sample. Modular implants can be structured as a scaffold that mimics one or more of a vascular network, a neural network and an optical network.

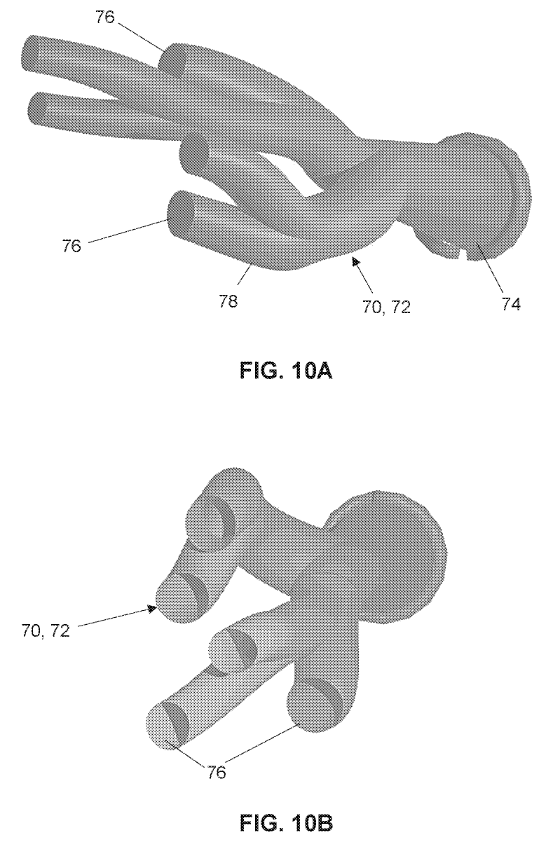

FIGS. 10A and 10B illustrate a modular implant 70 in the form of a vascular system having an inlet 74 and multiple outlets 76 through which fluid can flow within channels 78. In one embodiment, inlet 74 can be plugged into or otherwise connected to inlet port 80 for example as illustrated in FIG. 11. In an important aspect of the present invention cells can be grown or seeded on the modular implant such as the vascular systems illustrated in FIGS. 10A, 10B and 11 to facilitate various studies as desired by the operator or investigator.

Still further, if desired, each outlet 76 of modular implant 70, 72 can be connected to a separate outlet port 90 of the microfluidic cell device 10.

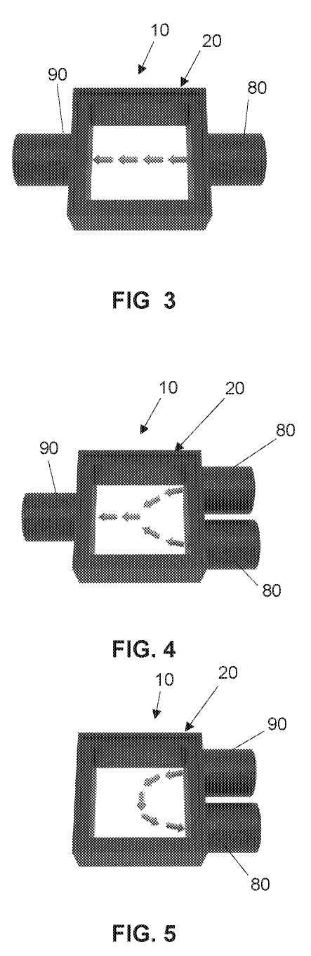

As mentioned above, construction of the main body and ports thereof are highly adaptable. For example, as illustrated in FIG. 3 cell device 10 includes inlet port 80 located on an opposite side of main body 20 as compared to outlet port 90. FIG. 4 illustrates two inlet ports 80 positioned on the same wall of main body 20 and opposite from outlet port 90. FIG. 5 illustrates cell device 10 including main body 20 having an inlet port 80 located in the same wall as outlet port 90.

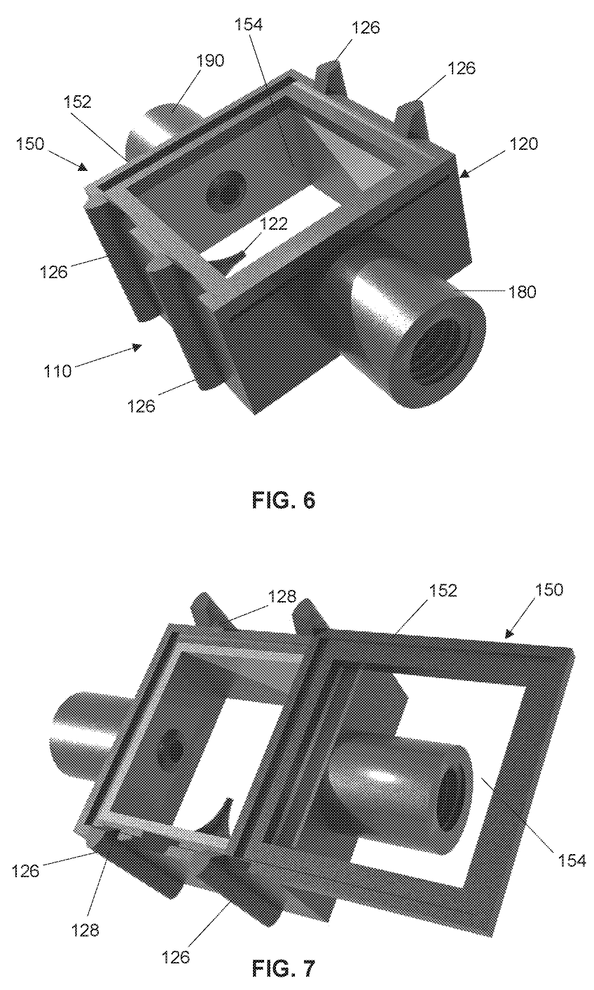

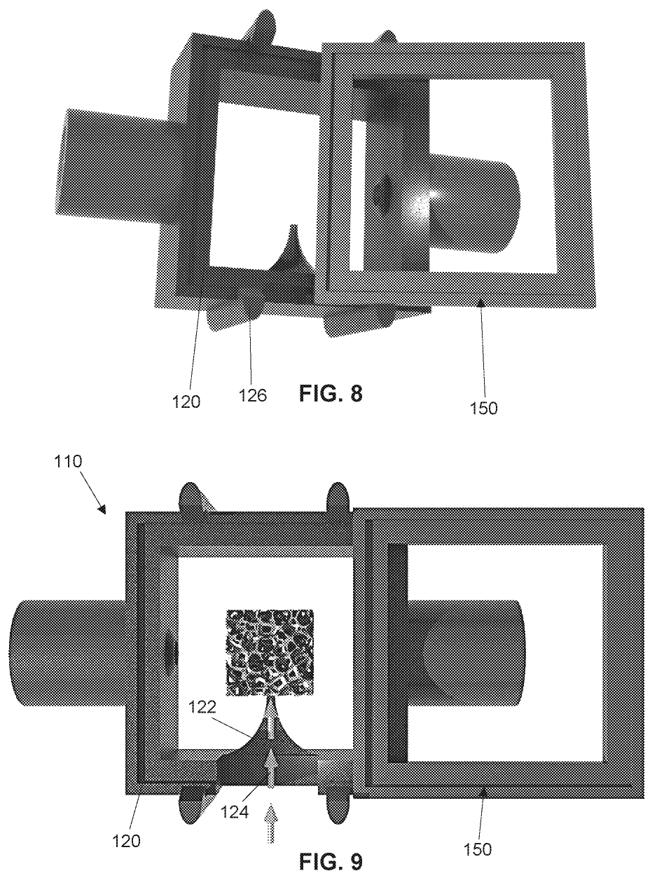

FIGS. 6-8 illustrate a second embodiment of a microfluidic cell device 110 having components analogous to microfluidic cell device 10 and thereby having a main body 120 comprising an internal, three-chamber dimensional chamber 130 having a volume.

Main body 120 includes a first window surface 140, namely the lower surface illustrated in FIG. 6. The first window surface is preferably substantially optically transparent.

Cell device 110 further includes a hatch 150 removably connected to the main body as illustrated between FIGS. 6-8. In the embodiment illustrated, main body 120 includes hatch locks 126 which secure hatch 150 to the main body. Hatch locks 126 as illustrated are in the form of posts having a recess or groove 128 that receive and retain hatch 150. The posts have a base connected to main body 20 and a head that extends outwardly past the main body and includes the groove 128. Hatch locks 126 seal hatch 150 against the upper surface of main body 120 via a pressure fit. Hatch 150 can be slid laterally in and out of recesses or grooves 128 to either close the cell device 10 or provide an opening in order to place or remove a sample and/or a modular implant into/from the chamber 130. In the embodiment illustrated, hatch 150 includes frame 152 formed of a different material as compared to hatch window 154, which is transparent or substantially transparent.

The main body includes a first hatch lock connected to a side surface of the main body and a second hatch lock connected to an opposite side surface of the main body which together cooperate to seal the hatch to the main body.

In a further embodiment, the first hatch lock and the second hatch lock each include a groove which receive a sidewall of the hatch and retain the hatch in sealing arrangement with the main body. In an additional embodiment, the main body includes two first hatch locks on a first side of the body and two second hatch locks on an opposite side which cooperate to secure the hatch to the main body when in use.

Cell device 110 includes an inlet port 180 and an outlet port 190. Additional inlet ports and outlet ports can be added if desired as described hereinabove. Cell device 110 also includes a pillar 122 that can be structured as described above for pillar 22.

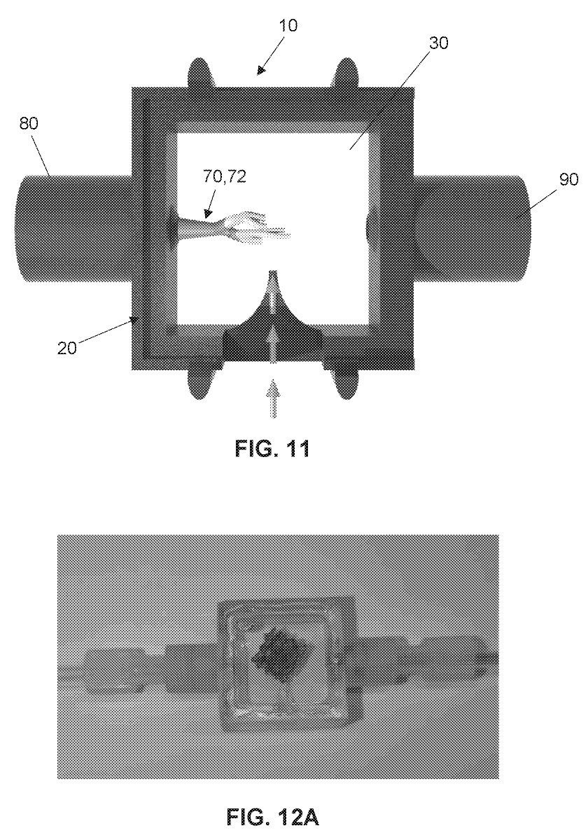

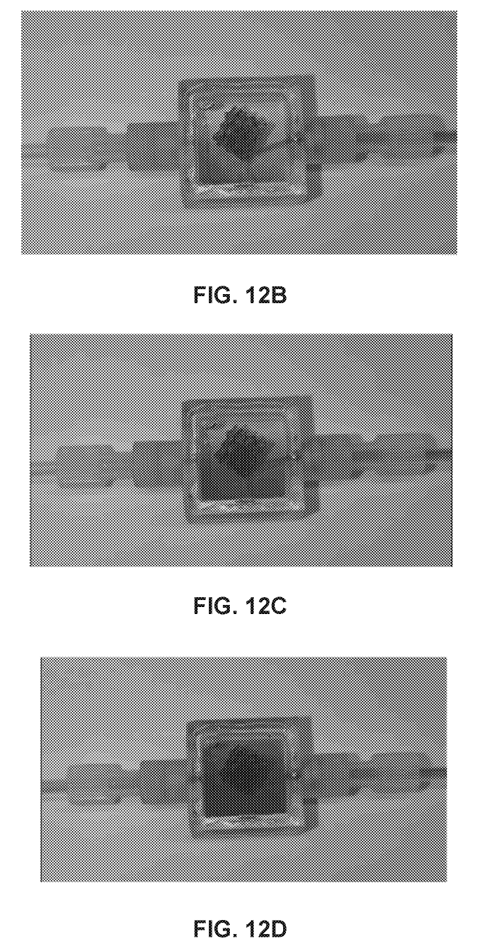

FIGS. 12A-12D serve to illustrate fluid flow through one embodiment of a cell device of the present invention. The microfluidic cell device is designed to allow flow of a material, namely a fluid such as a liquid or gas throughout a sample. In the time-sequential images shown in FIGS. 12A-12D one method of use of the cell device is illustrated. Starting from FIG. 12A, a cell device is illustrated having a sample situated on a pillar within the chamber. The main body includes an inlet port on the right side and an outlet port on the left side. Each port is connected to a tubing system containing the flowing media which is shown as dark material within the tubing inlet port on the right side in FIG. 12A. FIG. 12B shows the media entering the chamber from the inlet port on the right, wherein the chamber is approximately half-filled. FIG. 12C shows the chamber substantially completely filled with media which passes through the pillar-mounted sample before exiting the chamber on outlet port on the left side. FIG. 12D illustrates media flowing through the cell device from the inlet port through the outlet port and respective tubing connected thereto. In this way, a constant flow can continue throughout an experiment at any desired rate. In other iterations of the design, the number and locations of inlets and outlets may be customized to tailor the quantity of flowing materials and unique flow patterns desired.

1. A modular macrofluidic cell device, comprising: a main body having an internal, three-dimensional chamber having a volume and capable of housing a sample,
   wherein the main body further includes a first window surface, wherein the first window surface is sufficiently optically transparent such that when the sample is present in the chamber at least one observation can be made of the sample with an optical device through at least the first window surface,
   a hatch operatively and removably connected to the main body to allow placement of the sample into the chamber and removal of the sample from the chamber,
   wherein the main body has at least one inlet port extending through at least one wall of the main body between an outer surface of the wall and the chamber for introducing a fluid into the chamber, and
   wherein the main body includes at least one outlet port extending through at least one wall of the main body between an outer surface of the wall and the chamber for allowing the fluid out of the chamber.
2. The modular microfluidic cell device according to 1, further including a pillar having a base connected to the main body, wherein the pillar extends into the chamber, wherein the sample can be operatively connected to the pillar.
3. The modular macrofluidic cell device according to 2, wherein the pillar has a light path that extends from the base of the pillar and a distal second end of the pillar located a distance from the base and positioned in the chamber, and wherein the light path is able to transmit light from a light source located outside the chamber onto the sample when located in the chamber.
4. The modular macrofluidic cell device according to any of 1 to 3, further including a modular implant which is insertable into and removable from the chamber, and wherein the modular implant is adapted to serve as a substrate for the sample.
5. The modular macrofluidic cell device according to any of 1 to 4, wherein the at least one inlet port is present in a different wall of the main body as compared to the at least one outlet port.
6. The modular macrofluidic cell device according to any of 1 to 4, wherein the at least one inlet port is present in the same wall of the main body as compared to the at least one outlet port.
7. The modular macrofluidic cell device according to any of 1 to 6, wherein one or more of i) at least two inlet ports and ii) at least two outlet ports are present.
8. The modular macrofluidic cell device according to 4, wherein the at least one inlet port is located in a wall of the main body opposite the wall that includes the at least one outlet port.
9. The modular macrofluidic cell device according to any of 1 to 8, wherein the at least one inlet port is present in a wall perpendicular to the wall including the at least one outlet port.
10. The modular macrofluidic cell device according to any of 1 to 9, wherein one or more of the at least one input port and the at least one outlet port include a projection extending outwardly from the main body that can be capped to prevent fluid in the device from exiting the device through the respective inlet port and outlet port.
11. The modular macrofluidic cell device according to 4, wherein the modular implant is operatively connected to one or more of the at least one inlet port and at least one outlet port.
12. The modular macrofluidic cell device according to any of 1 to 11, wherein the modular implant comprises an artificial vascular system or scaffold.
13. The modular macrofluidic cell device according to any of 1 to 12, wherein the chamber is in the form of a cube, a cuboid, a cylinder, a sphere or a prism.
14. The modular macrofluidic cell device according to any of 1 to 13, wherein the hatch includes a second window surface, wherein the second window surface is sufficiently optically transparent such that when the sample is present in the chamber at least one observation can be made of the sample with an optical device through at least the second window surface, wherein second window surface is opposite the first window surface.
15. The modular macrofluidic cell device according to 12, wherein the scaffold mimics one or more of a vascular network, a neural network, and an optical network.
16. The modular microfluidic cell device according to any of 1 to 15, wherein the main body includes a first hatch lock connected to a side surface of the main body and a second hatch lock connected to an opposite side surface of the main body which together cooperate to seal the hatch to the main body.
17. The modular microfluidic cell device according to 16, wherein the first hatch lock and the second hatch lock each include a groove which receive a sidewall of the hatch and retain the hatch in sealing arrangement with the main body.
18. The modular microfluidic cell device according to 17, wherein the main body includes two first hatch locks on a first side of the body and two second hatch locks on an opposite side which cooperate to secure the hatch to the main body when in use.
19. The modular microfluidic cell device according to 4, wherein the modular implant includes an inlet which is operatively connected to the inlet port of the main body whereby a fluid exiting the inlet port flows through the modular implant.

For the avoidance of doubt, the devices and methods of the present invention encompass all possible combinations of the components, including various ranges of said components, disclosed herein. It is further noted that the term ‘comprising’ does not exclude the presence of other elements. However, it is also to be understood that a description of a product comprising certain components also discloses a product consisting of these components. Similarly, it is also to be understood that a description on a process comprising certain steps also discloses a process consisting of these steps.

In accordance with the patent statutes, the best mode and preferred embodiment have been set forth; the scope of the invention is not limited thereto, but rather by the scope of the attached claims.

What is claimed is:

1. A modular macrofluidic cell device, comprising:
a main body having an internal, three-dimensional chamber having a volume and capable of housing a sample,
wherein the main body further includes a first window surface, wherein the first window surface is sufficiently optically transparent such that when the sample is present in the chamber at least one observation can be made of the sample with an optical device through at least the first window surface,
a hatch operatively and removably connected to the main body to allow placement of the sample into the chamber and removal of the sample from the chamber,
wherein the main body has at least one inlet port extending through at least one wall of the main body between an outer surface of the wall and the chamber for introducing a fluid into the chamber, and
wherein the main body includes at least one outlet port extending through at least one wall of the main body between an outer surface of the wall and the chamber for allowing the fluid out of the chamber.

2. The modular microfluidic cell device according to claim 1, further including a pillar having a base connected to the main body, wherein the pillar extends into the chamber, wherein the sample can be operatively connected to the pillar.

3. The modular macrofluidic cell device according to claim 2, wherein the pillar has a light path that extends from the base of the pillar and a distal second end of the pillar located a distance from the base and positioned in the chamber, and wherein the light path is able to transmit light from a light source located outside the chamber onto the sample when located in the chamber.

4. The modular macrofluidic cell device according to claim 1, further including a modular implant which is insertable into and removable from the chamber, and wherein the modular implant is adapted to serve as a substrate for the sample.

5. The modular macrofluidic cell device according to claim 1, wherein the at least one inlet port is present in a different wall of the main body as compared to the at least one outlet port.

6. The modular macrofluidic cell device according to claim 1, wherein the at least one inlet port is present in the same wall of the main body as compared to the at least one outlet port.

7. The modular macrofluidic cell device according to claim 1, wherein one or more of i) at least two inlet ports and ii) at least two outlet ports are present.

8. The modular macrofluidic cell device according to claim 4, wherein the at least one inlet port is located in a wall of the main body opposite the wall that includes the at least one outlet port.

9. The modular macrofluidic cell device according to claim 1, wherein the at least one inlet port is present in a wall perpendicular to the wall including the at least one outlet port.

10. The modular macrofluidic cell device according to claim 1, wherein one or more of the at least one input port and the at least one outlet port include a projection extending outwardly from the main body that can be capped to prevent fluid in the device from exiting the device through the respective inlet port and outlet port.

11. The modular macrofluidic cell device according to claim 4, wherein the modular implant is operatively connected to one or more of the at least one inlet port and at least one outlet port.

12. The modular macrofluidic cell device according to claim 4, wherein the modular implant comprises an artificial vascular system or scaffold.

13. The modular macrofluidic cell device according to claim 1, wherein the chamber is in the form of a cube, a cuboid, a cylinder, a sphere or a prism.

14. The modular macrofluidic cell device according to claim 1, wherein the hatch includes a second window surface, wherein the second window surface is sufficiently optically transparent such that when the sample is present in the chamber at least one observation can be made of the sample with an optical device through at least the second window surface, wherein second window surface is opposite the first window surface.

15. The modular macrofluidic cell device according to claim 4, wherein the main body further includes a second window surface, wherein the second window surface is sufficiently optically transparent such that when the sample is present in the chamber at least one observation can be made of the sample with an optical device through at least the second window surface, wherein the second window surface is opposite the first window surface.

16. The modular macrofluidic cell device according to claim 12, wherein the scaffold mimics one or more of a vascular network, a neural network, and an optical network.

17. The modular microfluidic cell device according to claim 1, wherein the main body includes a first hatch lock connected to a side surface of the main body and a second hatch lock connected to an opposite side surface of the main body which together cooperate to seal the hatch to the main body.

18. The modular microfluidic cell device according to claim 17, wherein the first hatch lock and the second hatch lock each include a groove which receive a sidewall of the hatch and retain the hatch in sealing arrangement with the main body.

19. The modular microfluidic cell device according to claim 18, wherein the main body includes two first hatch locks on a first side of the body and two second hatch locks on an opposite side which cooperate to secure the hatch to the main body when in use.

20. The modular microfluidic cell device according to claim 4, wherein the modular implant includes an inlet which is operatively connected to the inlet port of the main body whereby a fluid exiting the inlet port flows through the modular implant.
